(19) United States
(12) Patent Application Publication
(10) Pub. No.: US 2012/0323322 A1
(43) Pub. Date: Dec. 20, 2012

(54) SURGICAL INSTRUMENT FOR CORPOREAL INTERNAL FIXATION SEPTOPLASTY

(76) Inventor: Oakley SMITH, Ontario (CA)

(21) Appl. No.: 13/494,365

(22) Filed: Jun. 12, 2012

Related U.S. Application Data
(60) Provisional application No. 61/497,136, filed on Jun. 15, 2011.

(51) Int. Cl. A61B 17/28; A61F 2/18

(52) U.S. Cl. 623/10; 606/205; 606/206

(57) ABSTRACT

A septoplasty instrument having a pair of opposing articulating longitudinal jaws or blades configured to contact the septum or overlying mucosal lining of a patient when the jaws are closed. A guide tube is connected to at least one of the jaws or blades for guiding a fixation wire into the septum, the guide tube having a bore therein that is generally in line with a longitudinal axis of the blades. The blades flatten and straighten a deviated portion of the septum, and the guide tube of the instrument is used to insert a fixation wire into the septum whilst the blades of the septoplasty instrument compressed. The instrument enables a corporeal approach to internal fixation septoplasty.

This application claims priority to U.S. Provisional Application No. 61/497,136, filed Jun. 15, 2011, the contents of which are incorporated herein by reference.

### FIELD OF INVENTION

The invention relates to the field of surgical instruments, and more particularly to an instrument in aid of septoplasty procedures.

### BACKGROUND OF INVENTION

Septoplasty is a surgical procedure for correcting a deformity of the nasal septum that causes the patient difficulty in nasal breathing. The deformity is typically characterized by a crookedness, curvature or deviation of the septum (the internal wall separating the two nostrils) that essentially blocks the opening to the nasal passages preventing normal nasal breathing. (FIG. 6B schematically illustrates a normal septum and FIG. 6A schematically illustrates a deformed septum requiring a septoplasty.)

The surgical goal of septoplasty is to create a straight septum and restore its position to the nasal midline. About 40% of these surgeries are successful, 30% partially successful, and 30% could be considered failures, with the more severe deformities characterized by higher failure rates. There is also a significant (greater than 5%) surgical revision rate.

Septoplasty techniques range from simpler, closed septoplasty techniques to open, extracorporeal approaches. Open approach septoplasty, typically used for severe septum deviations, involves exposing the bony septum by cutting at least some portion of the skin and cartilage overlying the bony septum. This technique provides maximal exposure and thus provides easy access for surgical maneuvers. However, it increases extracorporeal septoplasty the bony septum is literally removed from the patient so that the surgeon has the maximum freedom of maneuver to manipulate and shape the bone. It is desirable to minimize use of this procedure as it requires the most surgical time and increases rate of complications. It also typically prohibits the ability to carry out further nasal surgeries.

The techniques for straightening out a deviated septum are varied but one of the most promising—internal fixation septoplasty—utilizes one or more relatively stiff wires that are inserted into the bony septum and function as a framework for maintaining a straight septum. Internal fixation septoplasty currently requires the open extracorporeal approach to septoplasty due to the manipulations that are required. In particular, it is difficult to completely straighten a severely deviated septum corporeally to allow for a clean planar insertion of the fixation wires all the way through the septum without piercing the overlying mucosa, which is then prone to infection. Thus, in the open extracorporeal procedure the deviated bony septum that has been removed from the patient is literally flattened on the bench to enable the surgeon to insert the fixation wires through the plane of the septum following which the straightened septum is reattached. Even in this procedure it is difficult to manually install the fixation wires because the nasal septum has a wall thickness of about 2 mm and the fixation wires are about 0.5 mm thick leaving little room for error. The materials (bone and wires) are also relatively stiff making manual insertion difficult, increasing the risk of operator injury and increasing the risk of operator/patient cross infection. Furthermore, it is difficult to reattach the septum and realign the skin puncture especially given the new geometry of the straightened septum.

It would be desirable to be able to be able to carry out an internal fixation to straighten the septum corporeally, i.e., without the need for an open septoplasty approach, and so the invention seeks to provide an instrument that enables such a corporeal procedure, or at least expands the pool of patients for such a procedure.

### SUMMARY OF INVENTION

According to one aspect of the invention a surgical septoplasty instrument is provided that includes a pair of opposing articulating jaws, including a pair of opposing longitudinal blades configured to contact the septum or overlying mucosal lining of a patient when the jaws are closed. One of the blades is insertable into a first of two nostrils and the other blade is insertable into the second of two nostrils. A handle is connected to each jaw. A guide tube is connected to at least one of the jaws for guiding a fixation wire into the septum, the guide tube having a bore therein that is generally in line with a longitudinal axis of the blades.

Each blade preferably has a curved portion for circumnavigating the patient's columella and a straight portion for contacting septum. The straight portion of each blade preferably has a length sufficient to reach the patient's bony septum, and alternatively the blade length is sufficient to reach the anterior edge of the patient's bony septum.

In the preferred embodiment the straight portion of each blade carries a longitudinally split half of a wire guide thereon. The wire guide split halves are configured to pierce the mucosa and septum and define a bore therebetween when the jaws are closed, the bore being substantially in line with the guide tube. A plurality of such wire guides is preferred so as to provide a path for the fixation wire.

In the preferred embodiment the blades are transversely oriented relative to a plane defined by the handles and jaws.

The instrument preferably also includes a means, such as a leaf spring or spring clip, for biasing the jaws in an open position.

The guide tube may be formed as one piece and connected to one of the jaws, the other jaw having a cutout therein to accommodate the external periphery of the guide tube and allow the jaws to close flush. Alternatively, the guide tube may be separable from at least one of the jaws. Alternatively, the guide tube may be formed from two longitudinally split halves, one half being connected to one of the jaws and the other half being connected to the other jaw.

According to another aspect of the invention a surgical method is provided. The method include provisioning a septoplasty instrument having a pair of opposing articulating jaws including a pair of opposing longitudinal blades configured to contact the septum or overlying mucosal lining of a patient when the jaws arc closed, one of the blades being insertable into a first of two nostrils and the other blade being insertable into the second of two nostrils, a handle connected to each jaw, and a guide tube connected to at least one of the jaws for guiding a fixation wire into the septum, the guide tube having a bore therein that is generally in line with a longitudinal axis of the blades. The longitudinal blades of the septoplasty instrument are manipulated so as to compress and flatten or straighten a deviated portion of the septum. The guide tube of the septoplasty instrument is used to insert a fixation wire into the septum whilst the blades of the septoplasty instrument are compressed.

### BRIEF DESCRIPTION OF DRAWINGS

The foregoing and other aspects of the invention will be better understood with reference to the drawings, wherein:

FIG. 1 is a perspective view of a surgical instrument for carrying out a corporeal internal fixation septoplasty according a preferred embodiment, the instrument being shown in an open position;

FIG. 2 is a perspective view of the surgical instrument shown in FIG. 1 in a closed position;

FIG. 3 is a partial end view of one of two blades of the surgical instrument shown in FIG. 1;

FIG. 4 is a partial side view of the surgical instrument blade shown in FIG. 3;

FIG. 5 is a detail view of a portion of FIG. 3;

FIGS. 6A and 6B are schematic illustrations of a deviated septum and normal (straight) septum, respectively;

FIGS. 7A and 7B are side and front views, respectively, of a initial step in utilizing the surgical instrument shown in FIGS. 1-5 to guide a fixation wire into the septum;

FIGS. 8A and 8B are side and front views, respectively, of a first step in utilizing the preferred surgical instrument;

FIGS. 9A and 9B are side and front views, respectively, of a second step in utilizing the preferred surgical instrument;

FIG. 9C is a cross-sectional view of the preferred surgical instrument in operation taken from the perspective of the base of the nose;

FIGS. 10A and 10B are side and front views, respectively, of a third step in utilizing the preferred surgical instrument;

FIGS. 11A and 11B are side and front views, respectively, of a fixation wire installed in the septum to straighten the septum pursuant to the third step;

FIGS. 12A and 12B are side and front views, respectively, of a initial step in utilizing the surgical instrument shown in FIGS. 1-5 to guide a second fixation wire into the septum;

FIGS. 13A and 13B are side and front views, respectively, of a first step in utilizing the preferred surgical instrument to install the second fixation wire;

FIGS. 14A and 14B are side and front views, respectively, of a second step in utilizing the preferred surgical instrument to install the second fixation wire;

FIGS. 15A and 15B are side and front views, respectively, of a third step in utilizing the preferred surgical instrument to install the second fixation wire; and

FIG. 16 is a lateral view of corporeal internal fixation septoplasty wherein the preferred surgical instrument is utilized to install three fixation wires to straighten the septum.

### DETAILED DESCRIPTION OF PREFERRED EMBODIMENTS

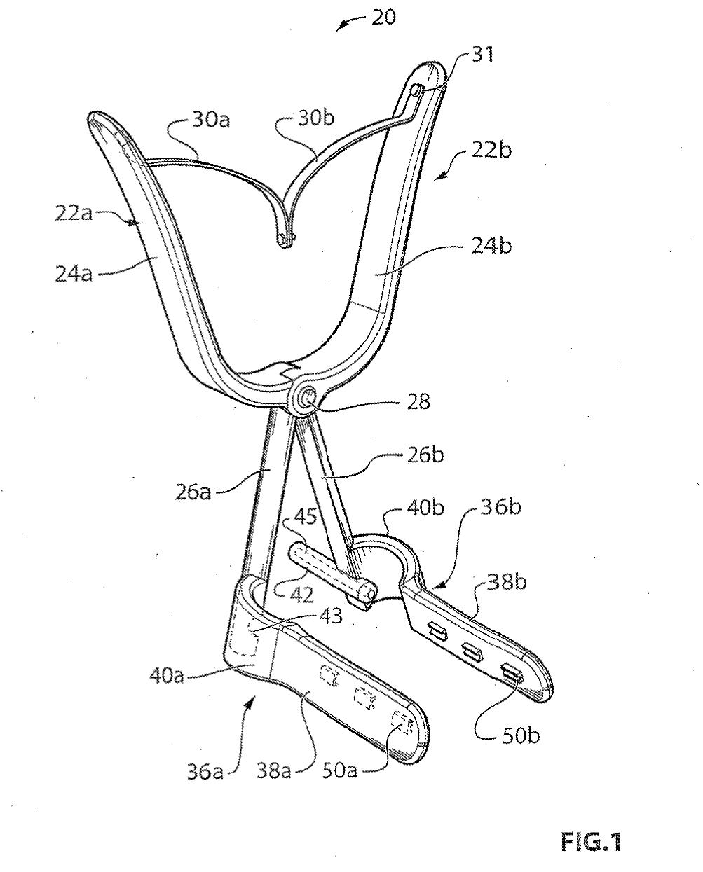

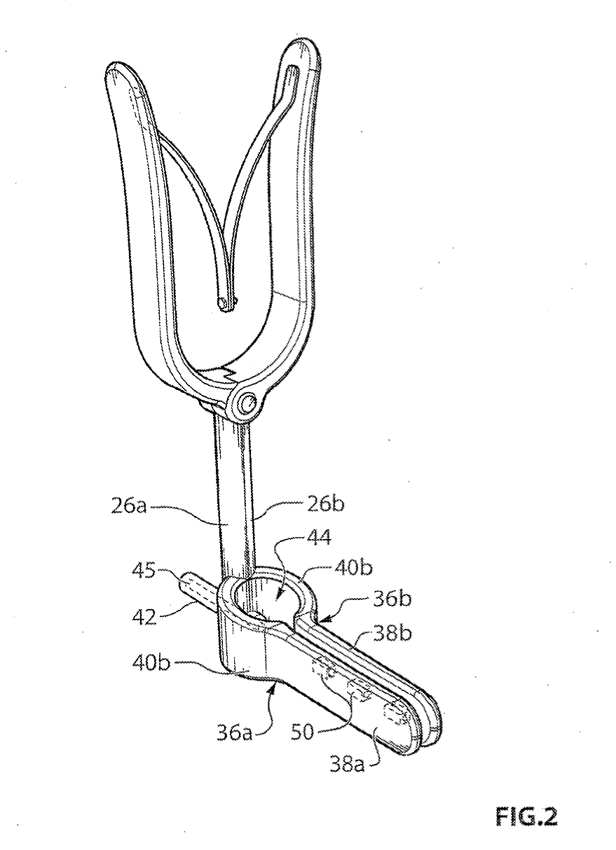

Referring to the perspective views of FIGS. 1 and 2 where the surgical instrument 20 according to the preferred embodiment is shown in open and closed views, respectively, it will be seen that the instrument 20 has a pliers-like body 22 including two pliers halves 22a, 22b comprising opposing handles 24a, 24b with integral depending jaws 26a, 26b extending generally along the same plane defined by the handles 24a, 24b. The pliers halves 22a, 22b are connected together by a pin or bolt 28 which enables the jaws 26a, 26b to articulate and more particularly pivot relative to one another about the pin or bolt 28. The pliers-like body 22 is biased to an open position as shown in FIG. 1 via two spring clips 30a, 30b, each of which has one end 31 connected to the inside of corresponding handle 24a or 24b and the other end 32 connected to the other spring clip. The pliers-like body 22 can be closed by the application of manual pressure and the jaws 26a and 26 have flat inside faces to enable them to close flush against one another, as seen in FIG. 2.

Two preferably integrally formed compression 36a, 36b are disposed at or near the end portions of the jaws 26a and 26b distal to the handles 24a, 24b. The blades 36a, 36b are preferably oriented transverse to the plane defined by the pliers halves 22a, 22b. The blades 36a, 36b may be perpendicular to this plane, or oriented at an angle thereto. Each blade 36a, 36b includes a straight portion 38a or 38b and a curved portion 40a or 40b proximate the corresponding jaw 26a or 26b. The straight blade portion 38a, 38b is intended and sized to be placed inside the nostril and the curved blade portion 40a, 40b is intended and sized to circumnavigate the fleshy base end of the septum (columella) so that the straight blade portions 38a, 38b can contact the mucosal lining (mucosa) overlying the septum and bear against the septum without interference by the base end of the nose.

The jaw 26b also includes a guide tube 42 that is oriented generally in line with the straight blade portion 38b. The opposing jaw 26a features a cutout 43 corresponding to the outer peripheral shape of the guide tube to enable the jaws 26a, 26b to close flush. The guide tube 42 has a bore 45 sized to permit passage of a fixation wire therethrough. As illustrated the guide tube 42 protrudes slightly into the space 44 (FIG. 2) defined by the curved portions 40a, 40b of the blades 36a, 36b but the extent of this protrusion may be cut back if desired or more preferably eliminated all together. The guide tube 46 extends rearwardly, in a direction opposite that of the blade 36b, and has a length and bore diameter sufficient to impart directional stability to the fixation wire as it is inserted into the septum and prevent the fixation wire from buckling. The bore 45 provides a relatively tight fit against the fixation wire so as to be able to support it longitudinally.

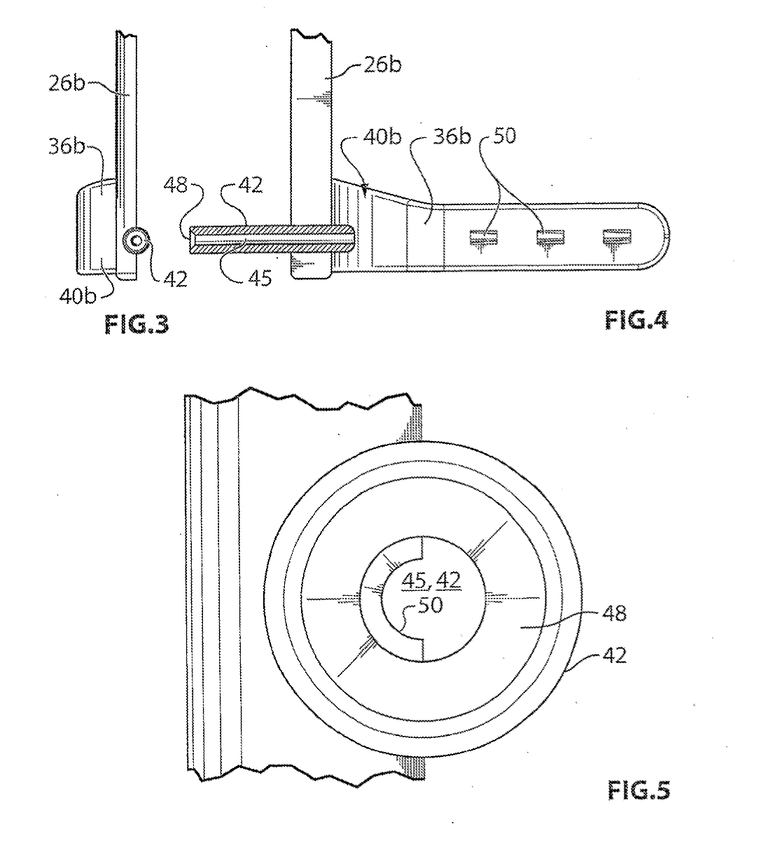

Referring to FIG. 2, it will be seen that the blades 36a, 36b when closed, feature a wire guide structure comprising a plurality of spaced apart guides 50. Referring additionally to FIGS. 3 and 4, which are partial end and side views of the guide tube 42 and the blade 38b it will be seen that blade 36b carries one longitudinally split half 50b of the wire guide 50, the other longitudinally split half 50a being carried by the other blade 36a as shown in FIG. 1. The bore 45 of the guide tube 42 is aligned with the bores 52 of the wire guides 50, and as will be explained in greater detail below the wire guides 50 are designed to pierce the septum and overlying mucosa and provide a means for guiding a fixation wire internally within the septum as the fixation wire is inserted in the guide tube 42 and pushed forwardly in the direction of the blades 36a, 36b.

Referring additionally to FIG. 5, which is a detail view of FIG. 3, the guide tube has a frusto-conically shaped head end 48. Likewise, when closed, each wire guide 50 preferably has a frusto-conical shaped bore 52 therein, the wider end being disposed at the end proximal to the guide tube 42. The frusto-conical arrangements help ease or transition the fixation wire into the guide tube and wire guide bores. Such structure may be varied in alternative embodiments, for example, by making the diameters of successive wire guide bores smaller (much like the successively narrower fishing line guides of a fishing rod), or eliminated all together.

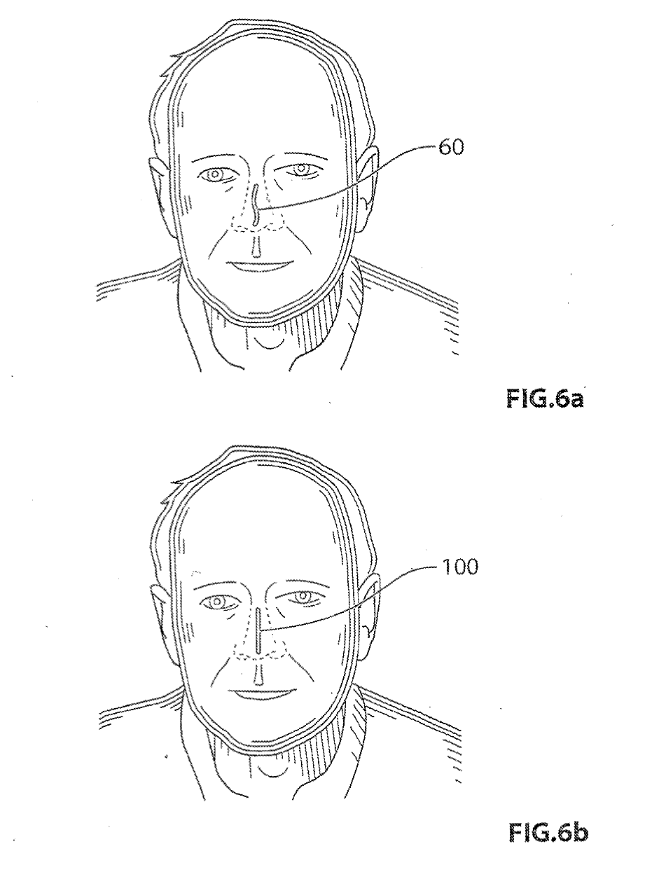

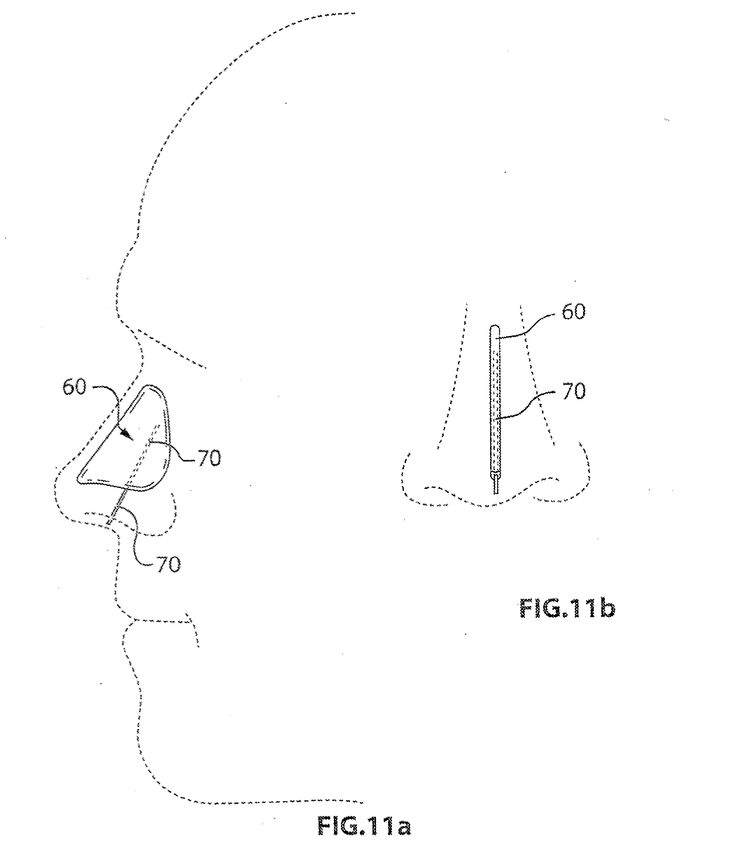

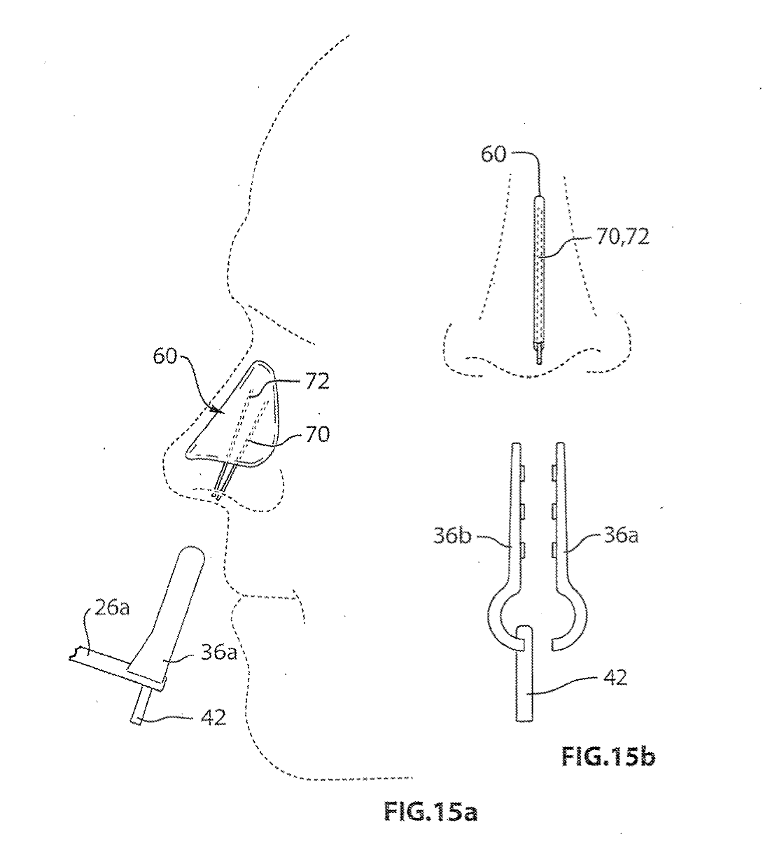

The method by which the surgical instrument is employed can be better understood with reference to FIGS. 6 through 15. FIG. 6A shows a patient with a deviated septum 60. FIG. 6B shows the desired end result of the corporeal internal fixation septoplasty—a healed straight septum 100, with the patient bearing no visible facial scars.

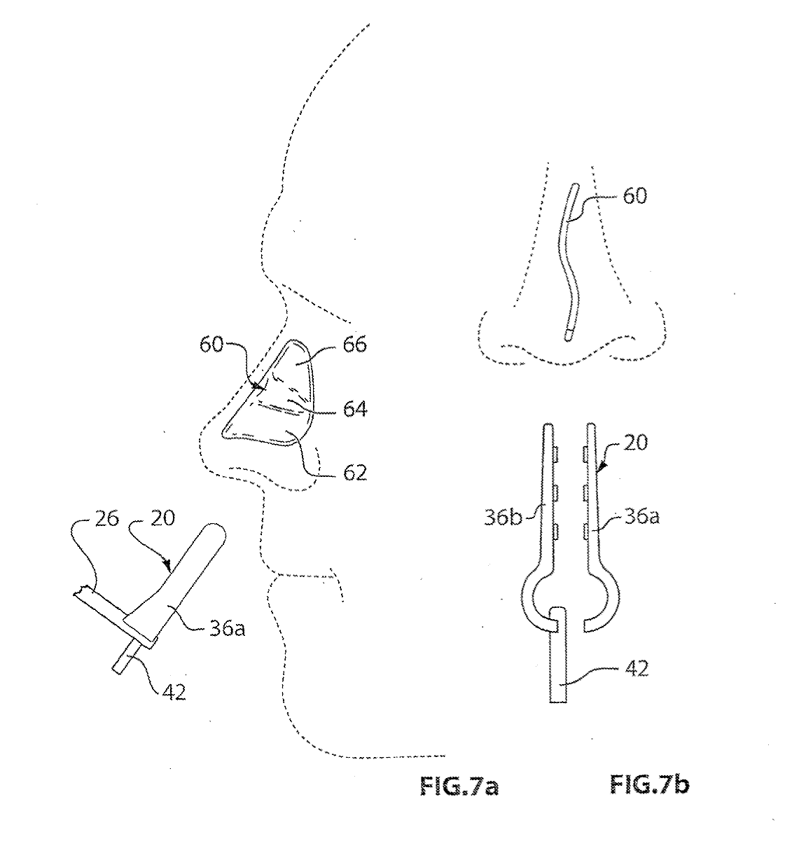

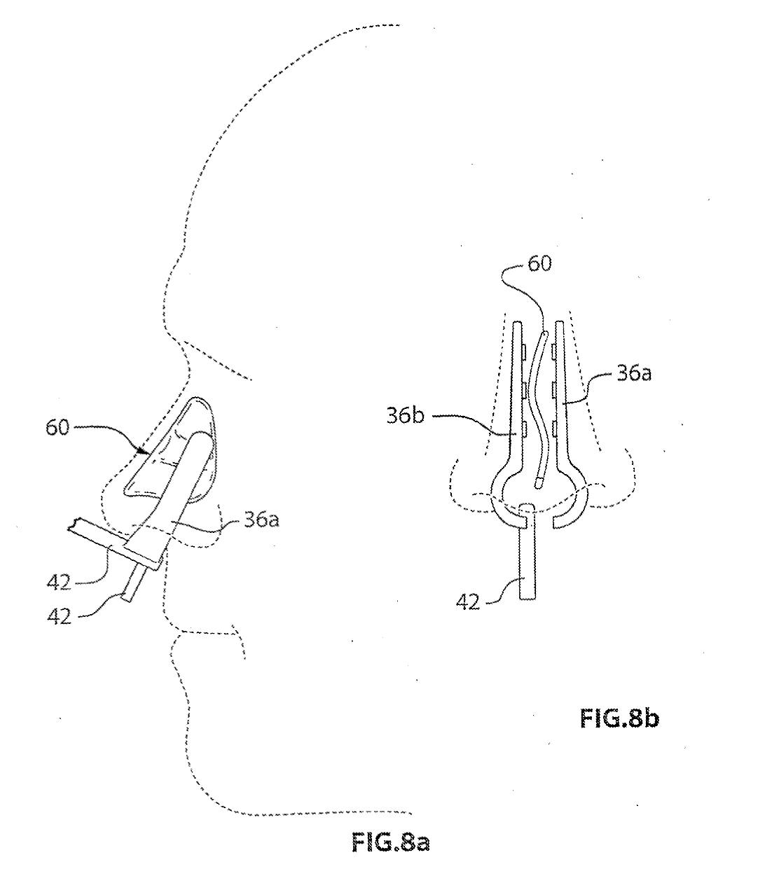

As seen in FIGS. 7A, 7B and 8A, 8B, the blades 36a, 36 of the surgical instrument 20 are guided into the nasal cavity so as to surround the septum. While different variants of the instrument 10 may have blades 36a, 36b of different lengths, the blades 36a, 36b are preferably at least long enough to extend across the cartilaginous septum 62 and reach the posterior edge of the bony septum 64, and more preferably extend to the anterior edge 66 of the bony septum 64. Using the nostril as a fulcrum, the blades 36a, 36b are manually oriented to straddle at least a portion of the nonlinear septum 60 as seen best in FIG. 8b. In this position it will be seen that the blades are not yet closed.

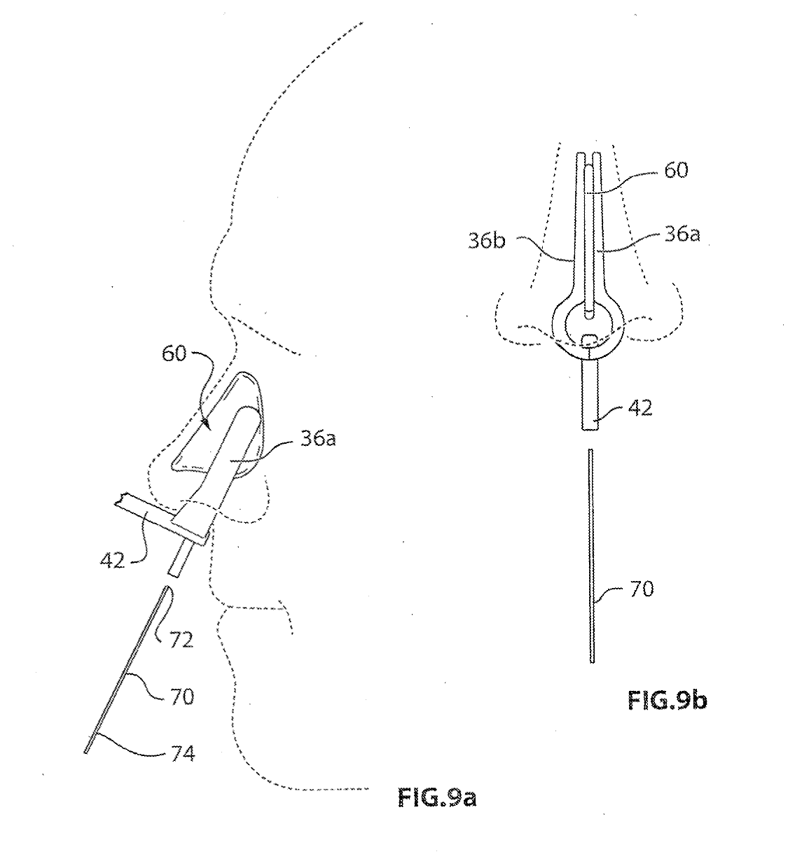

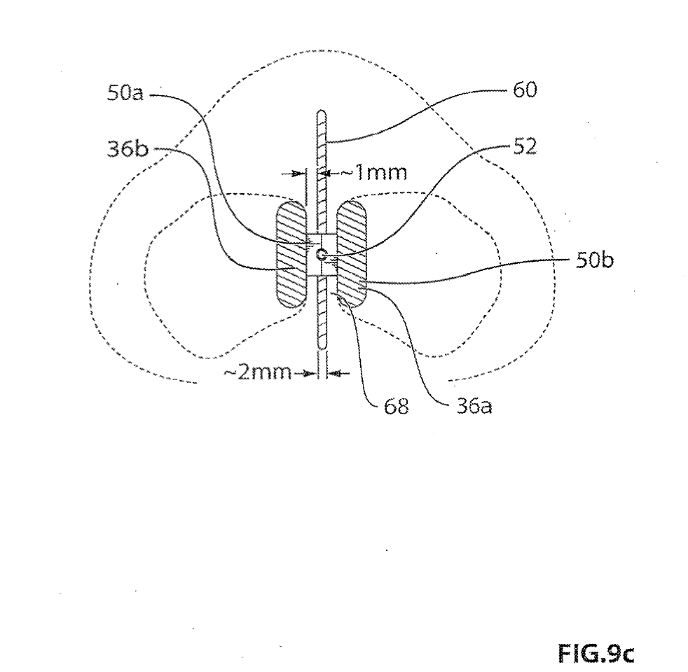

The blades 36a, 36b are shown closed in FIGS. 9A and 9B. Through the application of manual pressure on the handles, the blades are compressed to straighten out the nonlinearity of the septum. If desired or necessary, the surgeon may make an incision inside the nose across one side of the septum near the nostril opening to allow the surgeon to separate the mucosal lining from the septum on each side. The instrument can then be placed such that its blades 36a, 36b are between the septum and the inside of the mucosal lining. Alternatively, the incision may be omitted and the blades 36a, 36 positioned to the exterior of the mucosal lining. In addition, if desired or necessary, the cartilaginous or bony septum may also be cut or scored to provide relief incisions to allow the septum to fully straighten out.

The curved portions 40a, 40b of the blades 36a, 36b surround the fleshy and bulbous columella (not explicitly shown) at the base of the septum to enable the blades 36a, 36b to compress and straighten out the remainder of the septum. Where an incision is made that enables the surgeon to directly access the septum with the blades 36a, 36b, closing the handle tightly will compress the septum. Where no incision is made, closing the handle tightly will compress the mucosal lining and septum. As the blades are compressed, as seen in the schematic base view of FIG. 9C, the wire guide halves 50 will pierce the septum 60 (and mucosa 68 in the event it is also compressed) to matingly abut one another and form the wire guide bore 52 within the approximately 2 mm width of the septum. This minor piercing of the septum 60 (and mucosa 68 in the event it is also compressed) is not a major trauma, and will heal readily.

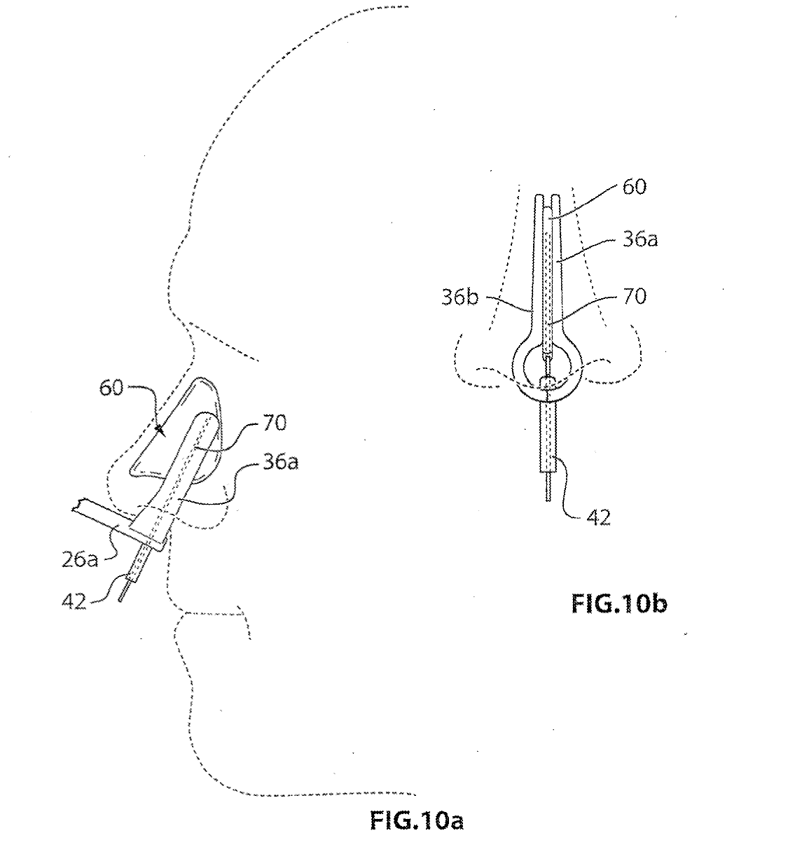

At this stage, the fixation wire 70 is introduced into the guide tube. The fixation wire 70 as known in the art per se has a sharpened end 72 for piercing the septum. The other end 74 of the wire 70 is connected to a drive unit as known in the art per se which is used to drive and/or drill the fixation wire into the septum, through the wire guides 50 of the blades 36 as shown in FIGS. 10A, 10B. The fixation wire 70 thus provides an anchoring pin or framework to hold and maintain straight the flattened portion of the septum compressed between the blades 36 of the instrument.

Once the fixation wire is embedded in the septum the instrument is removed. This may be accomplished in a variety of ways.

First, since the guide tube 42 is connected to only one of the blades 36b and the individual wire guides 50 are longitudinally split in half, the other blade 36a can be freely moved in the nostril even when the fixation wire is located in the guide tube. Given the inherent flexibility of the fixation wire 70, the first blade 36b can then be opened slightly somewhat and jiggled to remove the guide tube backward off of the embedded fixation wire.

Second, the fixation wire 70 may be accessed in the space 44 provided by the curved portions 40a, 40b of the blades 36 and clipped thereat with another tool such a needle-nosed snipper. As the individual wire guides 50 are longitudinally split in half, both blades 36a, 36b may be opened without dislodging the inserted fixation wire 70 and the blades 36a, 36b removed from the nostrils.

Third, the guide tube 42 may be a separable component as described in greater detail below, in which case the guide tube is slipped off the embedded fixation wire, enabling both blades 36a, 36b to freely open and be removed from the nose.

After the instrument is removed the installed fixation wire 70 maintains the septum in a linear alignment as the patient heals. Any excess length of the fixation wire may be clipped after installation. The fixation wire 70 will be removed after healing by simply pulling it through the tissue.

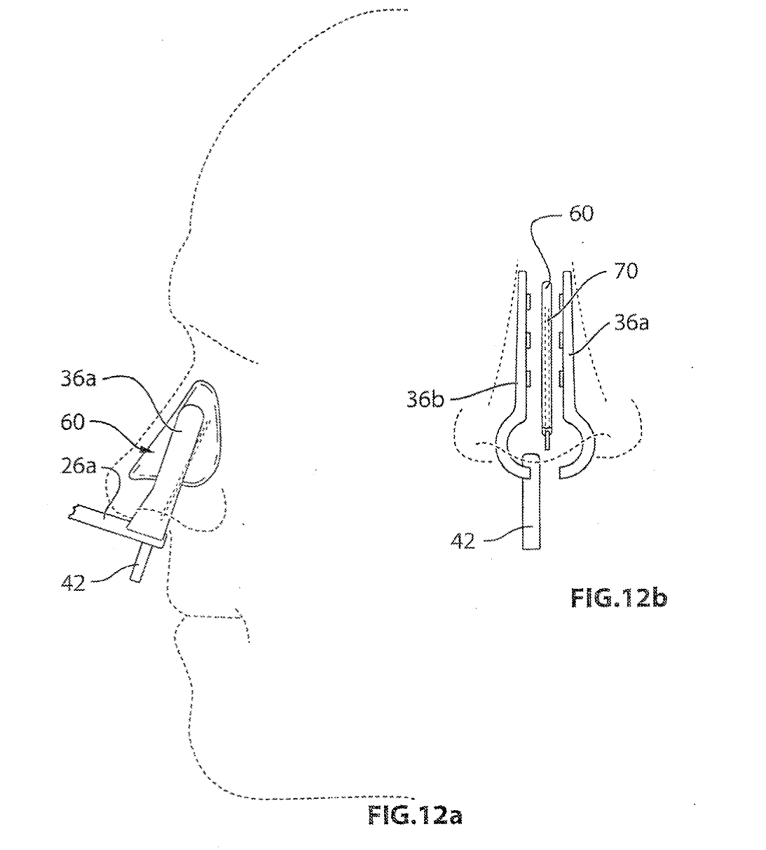

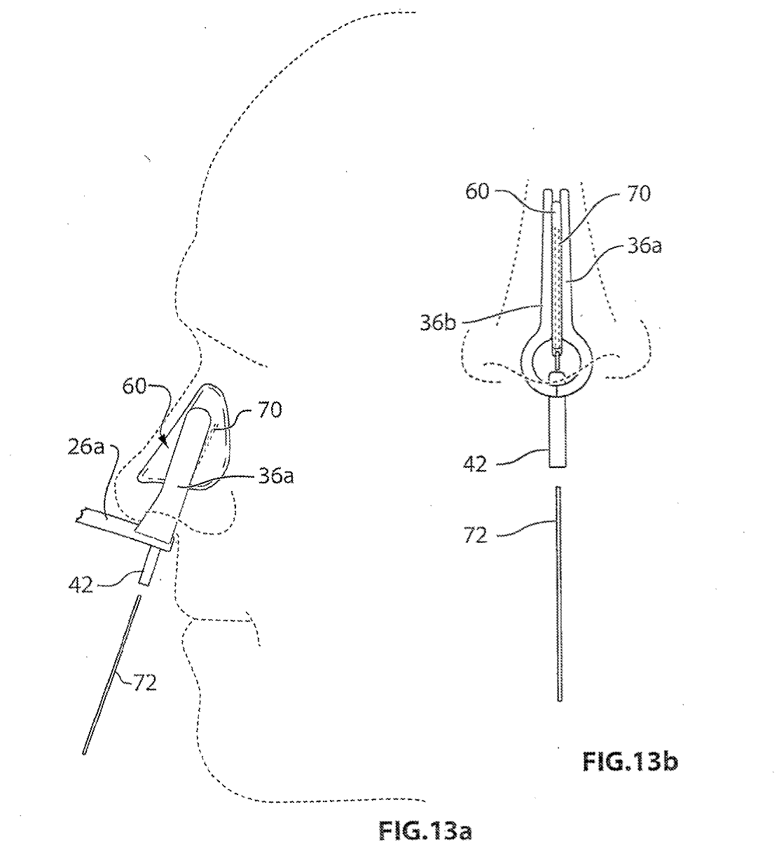

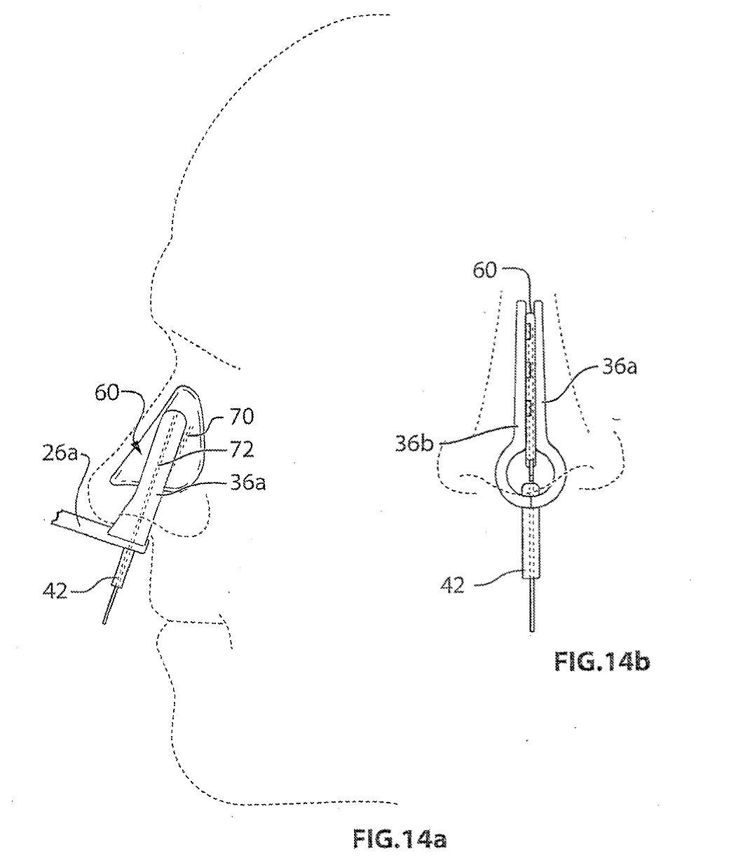

If desired or necessary, one or more additional fixation wires may be installed to straighten the septum and/or increase the rigidity of the septum during healing as shown in FIGS. 12-15. The same process outlined above is followed, but this time the instrument is manipulated using the base of the nostril as a fulcrum to orient the blades 36 at a different angle than the first instance as will be seen best in FIGS. 13A and 14A, enabling the second fixation wire 72 to be installed at a different angle than the first fixation wire 70.

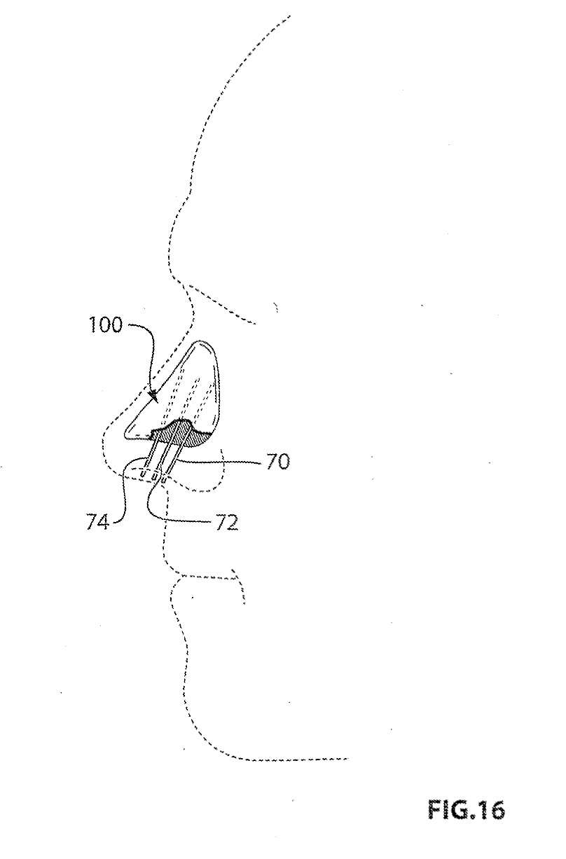

The fixation process may be repeated a number of times. FIG. 16 shows a corporeal internal fixation septoplasty in which the instrument 20 is utilized to insert three fixation wires 70, 72, 74 in order to provide a rigid framework for straightening out a severely deviated septum and maintaining it in a straight position whilst the patient heals.

In an alternative embodiment, the guide tube 42 may be provided as longitudinally split halves much like the wire guides 50, with one half of the guide tube attached to or otherwise carried by jaw 26a and the other half of the tube attached to or otherwise carried jaw 26b, the two halves mating together to form the bore 45 when the blades 36a, 36b are compressed. In this embodiment a long fixation wire may also be employed since the instrument 20 may be opened even if the fixation wire is located in the guide tube 42.

In another alternative embodiment the bore 45 within the guide tube 42 may be made larger than one sized to snugly accommodate the fixation wire 70. The larger bore may be employed to accommodate a chuck, collet or ferrule used to couple the fixation wire with its driver within the guide tube. In this case a shorter fixation wire may be employed and driven into the space 44 between the blades, after which the driver is removed. This too enables the instrument to be easily removed from the nose after the fixation wire is installed since no segment of the wire is left in the guide tube which would impede movement of the connected blade 36b.

In still other embodiments the guide tube 42 may be provided as a separable component that is screwed into a threaded opening provided in at least one of the jaws. Alternatively the separable guide tube 42 can be merely inserted into a bore, cutout or keyway formed in at least one of the jaws. For example, the keyway be formed as a dovetail in one of the jaws and the guide tube may have an external wall with a corresponding longitudinal dovetail profile in order to secure the separable guide tube with the jaw in a removable manner. In the foregoing manner the guide tube may be easily replaced with another as the need arises to accommodate, for example, various internal bore diameters and lengths.

In yet still another alternative embodiment the wire guides 50 may be omitted as the guide tube 42 may provide sufficient rigidity to the fixation wire in order to guide it along a straight path between the blades which also act as pincers to ensure that the fixation wire does not veer off course. Likewise while the preferred embodiment has shown the blades to be transverse and nearly perpendicular to the jaws, this is not absolutely necessary.

From the foregoing it should be appreciated that the invention enables corporeal application of internal fixation septoplasty, reducing trauma and healing times and eliminating visible facial scars arising from open surgical approaches. The invention is also envisioned to reduce surgical times, reduce the number of cases that would require open surgical approaches along with the technical challenges such an approach entails, and concurrently expand the pool of surgeons capable of carrying out septoplasty procedures.

Those skilled in the art may effect other alterations and modifications to the above-described embodiments, which are intended only to be examples of the invention, without departing from the spirit of the invention.

I claim:

1. A surgical septoplasty instrument, comprising:
a pair of opposing articulating jaws including a pair of opposing longitudinal blades configured to contact the septum or overlying mucosal lining of a patient when the jaws are closed, one of the blades being insertable into a first of two nostrils and the other blade being insertable into the second of two nostrils;
a handle connected to each jaw; and
a guide tube connected to at least one of the jaws for guiding a fixation wire into the septum, the guide tube having a bore therein that is generally in line with a longitudinal axis of the blades.

2. A surgical instrument according to claim 1, wherein each blade has a curved portion for circumnavigating the patients columella and a straight portion for contacting septum.

3. A surgical instrument according to claim 2, wherein the straight portion of each blade has a length sufficient to reach the patient's bony septum.

4. A surgical instrument according to claim 3, wherein the blade length is sufficient to reach the anterior edge of the patient's bony septum.

5. A surgical instrument according to claim 2, wherein the straight portion of each blade carries a longitudinally split half of a wire guide thereon, the wire guide split halves configured to pierce the mucosa and septum and defining a bore therebetween when the jaws are closed, the bore being substantially in line with the guide tube.

6. A surgical instrument according to claim 5, including a plurality of said wire guides so as to provide a path for the fixation wire.

7. A surgical instrument according to claim 1, wherein the blades are transversely oriented relative to a plane defined by the handles and jaws.

8. A surgical instrument according to claim 1, including a means for biasing the jaws in an open position.

9. A surgical instrument according to claim 1, wherein the guide tube is formed as one piece and is connected to one of the jaws, the other jaw having a cutout therein to accommodate the external periphery of the guide tube and allow the jaws to close flush.

10. A surgical instrument according to claim 9, wherein the guide tube is separable from at least one of the jaws.

11. A surgical instrument according to claim 1 wherein the guide tube is formed from two longitudinally split halves, one half being connected to one of the jaws and the other half being connected to the other jaw.

12. A surgical method, comprising:
providing a septoplasty instrument having a pair of opposing articulating jaws including a pair of opposing longitudinal blades configured to contact the septum or overlying mucosal lining of a patient when the jaws are closed, one of the blades being insertable into a first of two nostrils and the other blade being insertable into the second of two nostrils, a handle connected to each jaw, and a guide tube connected to at least one of the jaws for guiding a fixation wire into the septum, the guide tube having a bore therein that is generally in line with a longitudinal axis of the blades;
manipulating the longitudinal blades of the septoplasty instrument so as to compress and flatten a deviated portion of the septum; and
utilizing the guide tube of the septoplasty instrument to insert a fixation wire into the septum whilst the blades of the septoplasty instrument compressed.

13. A surgical method according to claim 12, including incising the mucosal lining so that the blades directly contact and compress the septum.

14. A surgical method according to claim 12, wherein each blade has a curved portion for circumnavigating the patient's columella and a straight portion for contacting septum.

15. A surgical method according to claim 14, wherein the straight portion of each blade has a length sufficient to reach the patient's bony septum.

16. A surgical method according to claim 15, wherein the blade length is sufficient to reach the anterior edge of the patient's bony septum.

17. A surgical method according to claim 12, wherein the straight portion of each blade carries a longitudinally split half of a wire guide thereon, the wire guide split halves configured to pierce the mucosa and septum and defining a bore therebetween when the jaws are closed, the bore being in line with the guide tube and assisting to guide the fixation wire into the septum when the blades are compressed and the wire guide halves meet up, the septum being punctured by the wire guides.

18. A surgical method according to claim 17, including a plurality of said wire guides in line with the guide tube so as to provide a path for the fixation wire.
